(19) United States
(12) Patent Application Publication
(10) Pub. No.: US 2021/0221861 A1
(43) Pub. Date: Jul. 22, 2021

(54) INDUCTION OF DNA STRAND BREAKS AT CHROMATIN TARGETS

(71) Applicant: Nzumbe, Inc., Portland, OR (US)

(72) Inventors: Michael Rodney Rountree, Portland, OR (US); James M. Stafford, Portland, OR (US)

(21) Appl. No.: 17/150,959

(22) Filed: Jan. 15, 2021

Related U.S. Application Data
(60) Provisional application No. 62/962,766, filed on Jan. 17, 2020.

(51) Int. Cl. C07K 14/47; C12N 9/22

(52) U.S. Cl. CPC C07K 14/4702; C07K 2319/80; C12N 9/22

(57) ABSTRACT

One aspect of this disclosure relates to a composition of matter. The composition of matter comprises a nucleotide construct encoding a peptide. The peptide includes at least a targeting domain configured to bind to chromatin having a pattern of reduced epigenetic repression, and a DNA strand break inducing domain. When accumulated through binding at chromatin sites, the strand break inducing domain may cause specific, double-strand breaks to the DNA, inducing cell death in cells exhibiting the pattern of reduced epigenetic repression.

### CROSS REFERENCE TO RELATED APPLICATIONS

This application claims priority to U.S. Provisional Application No. 62/962,766, filed Jan. 17, 2020, the entirety of which is hereby incorporated herein by reference for all purposes.

### BACKGROUND

Many cancer types are associated with aberrant epigenetic regulation. Tumor suppressor genes are often repressed through epigenetic downregulation, while growth and replication promoting genes are upregulated. Many cancer cell types develop similar epigenetic patterns that result in uncontrolled growth and dysregulation.

### BRIEF DESCRIPTION OF THE DRAWINGS

FIG. 1 schematically illustrates examples of permissive and repressive chromatin packaging.

FIG. 2 shows example constructs including methylation-sensitive DNA-binding domains coupled to DNA strand break inducing domains.

FIG. 3 shows an example construct including a modification-sensitive histone-binding domain coupled to a DNA strand break inducing domain.

FIG. 4 schematically shows example compositions of matter targeting a cancer cell.

FIG. 5 shows an example method for treating a tumor-bearing mammal.

FIG. 6 is experimental data showing the induction of DNA damage through targeting of hypomethylated LINE-1 elements.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

One aspect of this disclosure relates to a composition of matter. The composition of matter comprises a nucleotide construct encoding a peptide. The peptide includes at least a targeting domain configured to bind to chromatin having a pattern of reduced epigenetic repression, and a DNA strand break inducing domain. When accumulated through binding at chromatin sites, the strand break inducing domain may cause specific double-strand breaks to the DNA, inducing cell death in cells exhibiting the pattern of reduced epigenetic repression.

### DETAILED DESCRIPTION

This detailed description puts forth a therapeutic approach that targets common epigenetic changes that occur in many cancers, including changes in histone modification and loss of cytosine methylation (“hypomethylation”) at repetitive DNA elements. Exploiting this loss of epigenetic repression allows for treatments that precisely affect their function in aberrant cells, with limited activity in healthy, properly regulated cells.

Classic chemotherapeutic agents, while typically effective against cancer cells, are often plagued by “off-target” adverse effects on normal, healthy cells. Thus, a major goal in the development of new chemotherapeutic agents is to eliminate or minimize these “off-target” effects. One approach to achieving this goal is through targeting the very biochemical processes and/or events that distinguish cancer cells from normal cells. One example of such a difference is the disruption of normal DNA methylation patterns in most cancers. This dysregulation may take the form of both gains (hypermethylation) and losses (hypomethylation) of cytosine methylation at specific locations in the genome.

Gene specific hypermethylation in cancer is often found at DNA sequences associated with the promoters of tumor suppressor genes (TSGs). Aberrant hypermethylation has been implicated as an important part of an epigenetic cascade of events that results in the transcription of the hypermethylated TSGs being turned-off or “silenced”. Silencing of TSGs plays a significant and direct role in tumorigenesis.

Additionally or alternatively, most cancers show a global loss of DNA methylation, with the majority of this loss occurring in the repetitive DNA sequences that constitute a significant portion (e.g., ˜80%) of the human genome (e.g., Long Interspersed Nuclear Elements (LINEs), Short Interspersed Nuclear Elements (SINEs) such as Alu sequences, LTR retrotransposons, non-LTR retrotransposons, DNA transposons, pericentromeric repeats, etc.). This hypomethylation of repetitive DNA sequences is believed to result in chromosomal instability and increased mutational events that help drive tumorigenesis. Thus, both aberrant hypermethylation and hypomethylation present “tumor signatures”. Aberrant methylation signatures may be used to identify cancer cells, while hypomethylation signatures may be therapeutically exploited using novel, targeted approaches to damage or kill cancer cells.

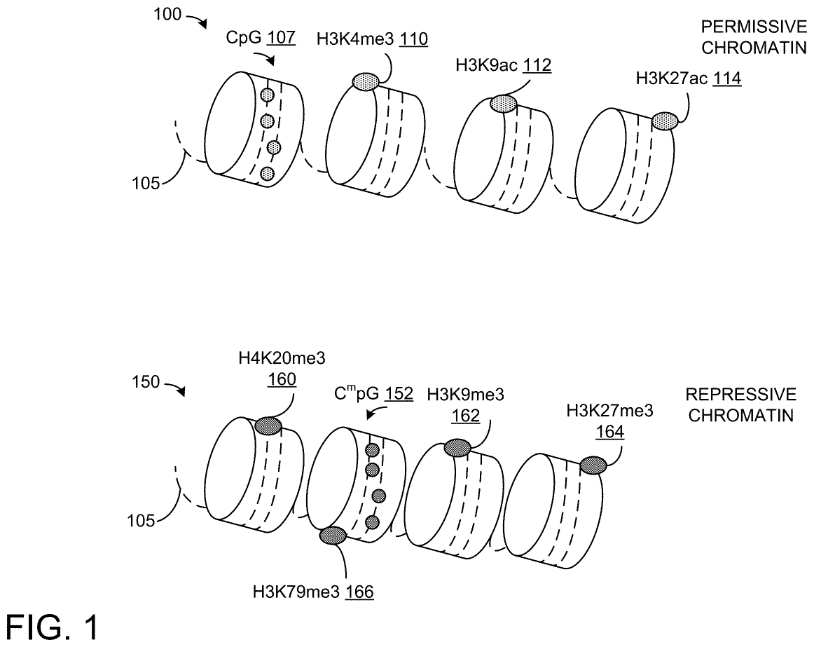

FIG. 1 shows DNA within cells is packaged as chromatin, a dynamic structure composed of nucleosomes as the fundamental building blocks. Histones are the central component of the nucleosome, forming an octamer containing the four core histone proteins (H3, H4, H2A, H2B) around which is wrapped a ˜147-base-pair segment of DNA. Each histone protein possesses a characteristic amino-terminal tail, which includes numerous lysine and arginine residues. The histone tails are subject to extensive posttranslational modifications, particularly on these basic residues. The modifications, along with methylation of cytosine residues within CpG dinucleotides of the DNA cooperate to govern the state of the local chromatin.

Broadly, chromatin exists in active/permissive and restrictive/repressive states. Examples of these states are shown in FIG. 1. At 100, four histones (102) are shown wrapped in DNA (105, dashed line) in a permissive state. Therein, the chromatin is open (euchromatin), allowing for transcription factors and other binding agents to target DNA sequences. DNA 105 includes unmethylated CpG dinucleotides 107. Representative histone modifications indicative of transcriptionally active chromatin are shown, including H3K4me3 (110), H3K9ac (112), and H3K27ac (114).

At 150, histones 102 and DNA 105 are shown in a repressive state. The chromatin is condensed (heterochromatin), preventing the binding of transcription factors. DNA 105 includes methylated mCpG dinucleotides 152. Representative histone modifications indicative of transcriptionally inactive chromatin are shown, including H4K20me3 (160), H3K9me3 (162), H3K27me3 (164), and H3K79me3 (166). These differences may be exploited to target cancer cells and/or other cells with aberrant epigenetic regulation. By targeting chromatin having repetitive patterns of reduced epigenetic repression, such as those having DNA sequences and histone modifications associated with aberrant epigenetic signatures, “normal” cells may be left alone, allowing for precise targeting.

This description provides methods and compositions of matter designed to target and cleave hypomethylated, repetitive DNA sequences in cancer cells. This may be accomplished using methylation-sensitive, sequence specific DNA binding agents and/or agents specifically targeting histone moieties associated with active chromatin. Such targeting agents may be coupled to DNA strand break inducing agents, such as transcription activator-like effector nucleases (TALEN) or other targeted DNA nucleases/machinery, such as those that cleave DNA in a methylation-sensitive manner. The resulting genome-wide double strand breaks (DSBs) induced in targeted cancer cells is intended to trigger their death through the process of apoptosis or other cell death machinery.

As one example, hypomethylation-induced target-mediated apoptosis (HITMA) may be used to specifically target and induce DSBs in hypomethylated, repetitive DNA elements in cancer and other diseases. Agents and compositions that initiate HITMA (HITMA agents) may target and bind to specific sequences in chromatin associated with these hypomethylated repetitive elements with significant specificity when compared to the same sequences when they are properly methylated in normal cells. In this way, the induction of apoptosis may be many-fold higher in cancer cells vs normal cells.

As used herein, the term “methylation-sensitive” refers to a peptide or nucleic acid whose binding affinity for a target DNA sequence is altered by DNA (e.g., cytosine) methylation and/or the histone modifications and/or other underlying chromatin structure(s) typically associated with DNA methylation. In most of the examples herein, “methylation-sensitive” indicates the inhibition of and/or a significant reduction of binding by such agents to methylated DNA vs unmethylated DNA. However, in some examples “methylation-sensitive” may refer to agents that have a higher binding affinity for a methylated DNA sequence (e.g., methylation-affinitive).

Many genotoxic anticancer drugs (e.g., bleomycin, etoposide, camptothecin) and treatments (e.g., ionizing radiation) induce DSBs, a type of DNA lesion that is particularly cytotoxic because it is so difficult to repair. The accumulation of DSBs triggers a cascade of events leading to apoptosis (programmed death) of cells. However, most of these anti-cancer treatments also cause adverse off-target effects on normal cells. Additionally, some are difficult to use for certain cancer types, and many require the co-administration of other medications or treatments that may further damage normal cells.

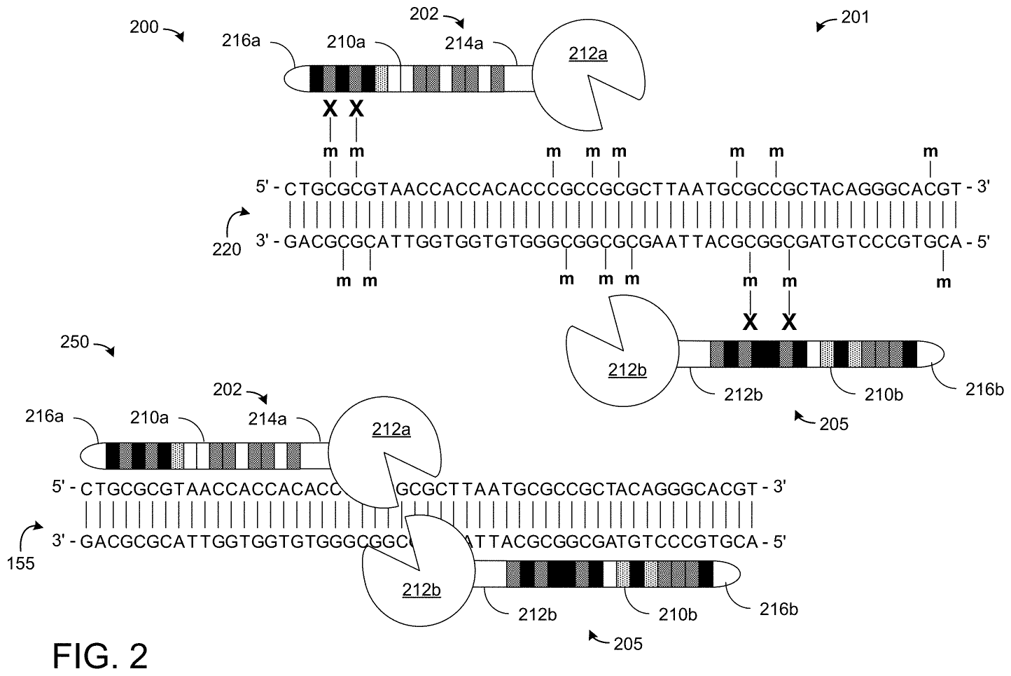

FIG. 2 shows an example composition of matter that may be used to induce targeted methylation-sensitive double-strand breaks in cancer cells. At 200, a composition 201 is shown comprising a pair of peptide molecules (202, 205), each having a targeting domain (210a and 210b), physically coupled to a DNA strand break inducing domain (212a and 212b). Each peptide molecule is further shown to include a linkage domain (214a and 214b) between the respective targeting domain and DNA strand break inducing domain, and a tail domain (216a and 216b). Each tail domain may serve to purify, stabilize, target, or otherwise aid the function of the peptide molecule.

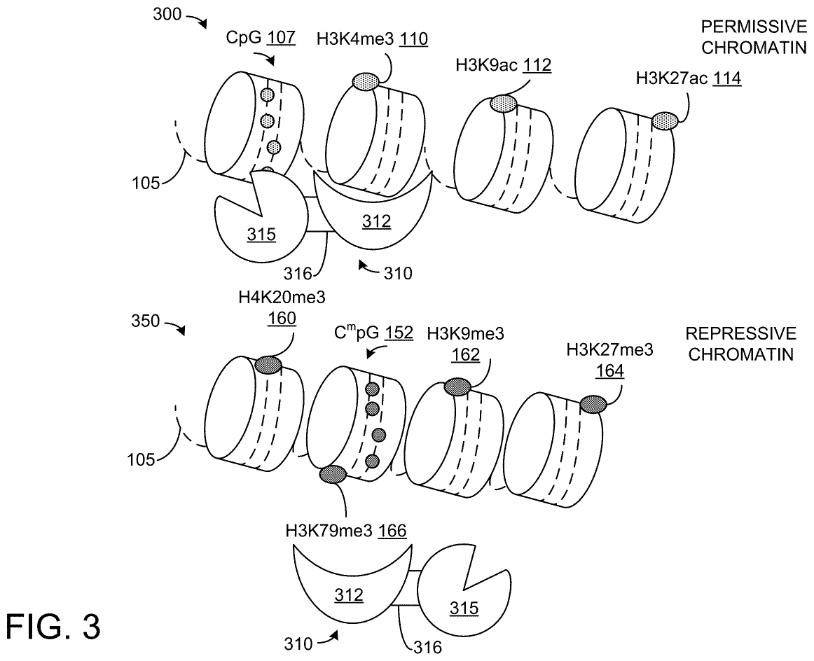

As an example, composition 201 may be included in a class of agents comprising transcription activator-like effector nucleases (TALEN), which are artificial nucleases that include a customizable DNA-binding domain and a nuclease domain such as the nuclease domain of the FokI restriction endonuclease enzyme. However, targeting domain 210a may include any suitable targeting domain, (e.g., DNA, RNA, and/or peptide based) which recognizes a target DNA sequence and is sensitive to DNA methylation of its recognition sequence. The recognition sequence may be associated with a repetitive element, and may have a cancer-specific hypomethyation pattern. Targeting domain 210b may be configured to bind at a neighboring sequence within a threshold distance of targeting domain 210a, so as to induce double-strand breaks. For example, targeting domains 210a and 210b may bind to sequences on opposite DNA strands, so as to induce strand breaks on both strands within a threshold number of base pairs. Additional examples, where the targeting domain binds to histone or other protein-based chromatin structures and modifications are described herein and with regard to FIG. 3.

Similarly, DNA strand break inducing domains 212a and 212b may comprise any suitable DNA strand break inducing agent (e.g., nuclease, restriction enzyme, chemical agent, nanomachine, catalytic RNA). In some examples, the strand break inducing domain may include one or more chemical agents, biochemical agents, mechanical agents (e.g., DNA clipping nanomachines), biomechanical agents, and/or other biological agents (e.g., peptide nuclease domains, catalytic RNA) that are capable of generating single strand or double strand breaks when brought into the proximity of a DNA molecule. In some examples, the DNA strand break inducing agent may be sensitive to DNA methylation (e.g., methylation-sensitive restriction enzyme domain).

Targeting domains 210a and 210b may be designed to target virtually any sequence motif and may be sensitive to DNA methylation at its recognition sequence. For example, at 200, a methylated DNA sequence 220 is shown. The methylated cytosine residues prevent the binding of targeting domains 210a and 210b. As the strand break inducing domains 212a and 212b are not bound in proximity to the DNA, no DSBs are generated in the repetitive sequence. However, at 250, a hypomethylated repetitive sequence 255 enables the binding of targeting domains 210a and 210b to their respective recognition sequences. The DNA strand break inducing domains 212a and 212b are then positioned at DNA sequence 255 in close enough proximity (e.g., within a threshold distance) so as to generate double-strand breaks in repetitive DNA sequence 255 when each strand is broken.

Targeting domains 210a and 210b may bind to the same, repetitive sequence motif or different sequence motifs, such that the binding of the domains to the sequence pairs the nuclease domains within the threshold distance of each other. For example, the high specificity of the DNA-binding domain and the ease of design have enabled researchers to use TALENs for targeted genome editing in various organisms. To generate a DSB in the DNA, two TALEN monomers may be used—one to bind the top (Watson) strand of the DNA and the second to bind the bottom (Crick) strand of the DNA with a 15-30 base pair spacer between, as shown at 250. By targeting repetitive sequences, numerous DSBs may be generated throughout the genome, which may be more likely to trigger the onset of apoptosis.

Thus, HITMA may apply the design of the DNA-binding domain regions of each TALEN monomer to target properly spaced recognition sequences in a repetitive DNA sequence. These recognition sequences may contain one or more CpG dinucleotides wherein the cytosine (C) is typically methylated in normal cells, but aberrantly hypomethylated in cancer. Different repetitive elements show variable aberrant hypomethylation in different cancer types/subtypes, so it is likely different HITMA-TALENs, and perhaps combinations of targeting domains and double-strand break inducing domains, would be designed to specifically target each cancer types and subtypes.

In another example, FIG. 3 shows an example peptide construct including modification-sensitive histone-binding domains coupled to DNA strand break inducing domains. At 300, a peptide construct 310 is shown, including a modification-sensitive histone-binding domain 312 coupled to a DNA strand break inducing domain 315 via a linker region 316. Using the example chromatin constructs from FIG. 1, modification-sensitive histone-binding domain 312 may bind to histones within permissive chromatin 114, such as histones featuring unmodified H3K79 moieties. As such, at 300, peptide construct 310 may bind to a histone, bringing DNA strand break inducing domain 315 into proximity to DNA 105, whereby DNA strand breaks may be induced.

In contrast, at 350, with repressive chromatin featuring H3K79me3 moieties, peptide construct 310 may not bind to a histone via modification-sensitive histone-binding domain 312, and thus DNA strand break inducing domain 315 is unable to act on DNA 105. In other examples, other moieties, such as histone H3K9 methylation may be used to distinguish between histones. In some examples, the modification-sensitive histone-binding domain may be paired with a methylation-sensitive DNA binding domain and/or strand break inducing domain, thereby providing an additional layer of protection for healthy chromatin. A composition may include two or more peptides, with multiple, different modification-sensitive histone-binding domains and/or methylation-sensitive DNA binding domains represented. As such, multiple DNA strand break inducing domains may be positioned in proximity to each other, increasing the likelihood of generating double strand breaks.

Numerous variations to the HITMA approach are discussed herein, but these are not intended to be limiting variants. The HITMA-TALEN constructs could be modified in any number of ways. For example, recent studies have reported that heterodimerization of modified FokI domains, ELD and KKR, increases nuclease activity. In scenarios wherein a properly spaced palindromic sequence motif can be identified in the repetitive sequence to be targeted, the use of only a single TALEN monomer would be possible. It has further been reported that when the nonspecific endonuclease, FokI, is replaced with a sequence-specific I-TevI homing endonuclease then DSBs can be induced with a TALEN:I-TevI monomer. This approach may work for other homing endonucleases that function as monomers, but may not work with classic TypeII restriction enzymes, as these typically work as dimers. The drawback is the requirement for the specific recognition sequence of the endonuclease to be within the target sequence. The platform further allows the flexibility for engineering other methods of HITMA targeting such as those specified below that may include, but are not limited to, altering the DNA-methylation sensitivity domains of the agents, altering regions that facilitate allosteric activation of nuclease activity, DNA-targeting specificity, etc.

In some examples, an agent other than TALENs may be used to target endonucleases to hypomethylated repetitive sequences. As one example, restriction enzymes (RE) or other endonucleases may be used. There are numerous examples of REs that are sensitive to methylated cytosine(s) within the target sequence. However, the recognition sequence for most REs are short and not specific to repetitive sequences. Significant off-target cutting may occur at other genomic sequences in both cancer and normal cells. REs, most likely methylation-sensitive ones, may be tethered to other proteins (TALs, zinc finger proteins, “enzymatically dead” CAS9 (dCAS9), DNA binding domains, etc.) that could direct them to specific sequences and this tethering may be an example of a successful approach to target and induce DSBs at aberrantly hypomethylated sequences in cancer.

Meganucleases can be engineered to target a specific sequence, but this protein engineering is much more difficult than engineering TALENs. Meganucleases have been reported to have some sensitivity to DNA methylation dependent on where the methylated cytosine falls within its recognition site. This may represent a good approach if the protein engineering challenges can be overcome.

Clustered Regularly Interspaced Short Palindromic Repeats (CRISPR) represents a technology that can be targeted to specified sequences but has a greater potential for off-target effects then the use of TALENs. CRISPR is not sensitive to DNA methylation of the guide RNA (gRNA) target sequence, but there is some evidence that higher-order chromatin structure, which is typically associated with DNA methylation, can inhibit its access. The effectiveness of this approach could be easily tested in cell lines (cancer vs normal). CRISPR could also potentially be utilized in HITMA-based methods as a mechanism to modify the gRNA nucleotides in such a way that would reduce or inhibit the ability of the gRNA to hybridize to methylated DNA sequences.

Zinc-Finger Nucleases (ZFN) can be designed to target virtually any sequence motif, but are currently not sensitive to DNA methylation. However, modifying zinc-finger binding domains in such a way to make the DNA binding domain sensitive to DNA methylation may provide an additional option for implementing the HITMA approach.

Combinatorial “Boolean-logic” DNA and methylation state-specific targeting may be used to enhance specificity and some of the efficiency of the system. In some examples, a delivery system may be employed wherein the methylation specific DSB-agent and the DNA sequence-specific targeting agent are added in parallel instead of being combined in the same agent. In this system, the methylation-sensitive nuclease or DSB-inducing agent may be engineered such that its activation is contingent on the presence of the recruitment of the DNA sequence-specific agent. This would allow the introduction of multiple DNA sequence specific agents into a system where the methylation sensitive DSB agent is present. This type of system may have numerous advantages, including, but not limited to: 1) more ease/flexibility in the number of sequences that can be targeted simultaneously via an individual vehicle for DNA specific targeting; 2) dividing the HITMA components into smaller delivery vehicles that may enhance delivery; 3) added safety by separating the DSB effector and its activator into separate vehicles.

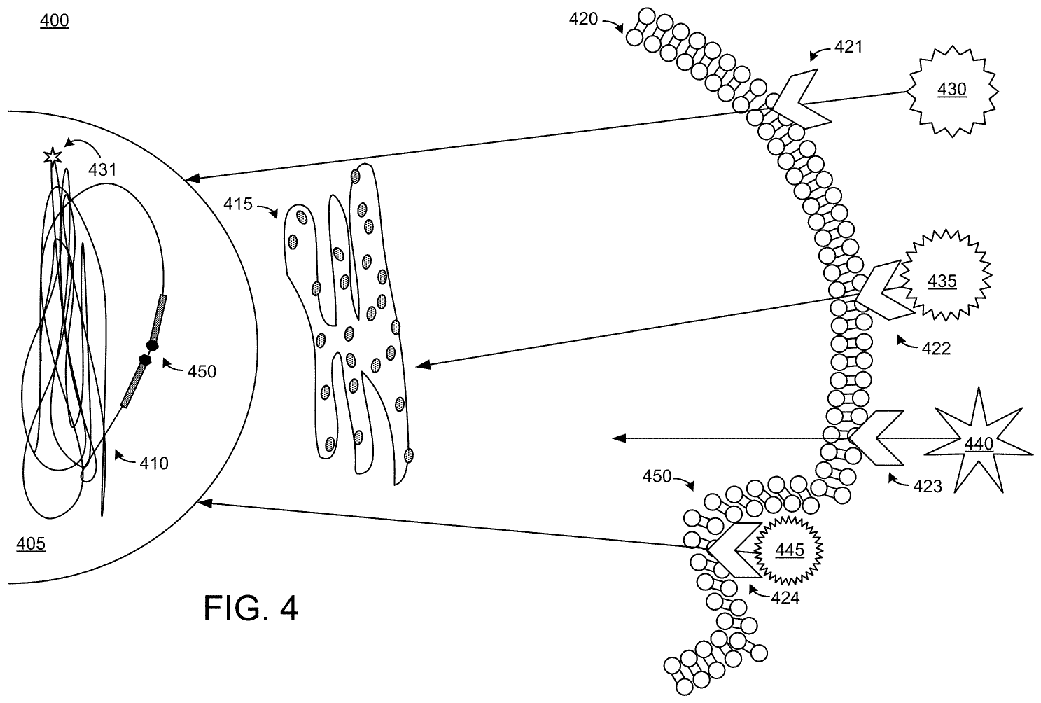

FIG. 4 schematically shows an example cancer cell 400 that includes at least a nucleus 405, a genome 410, an endoplasmic reticulum 415, and a cell membrane 420 expressing at least cell surface receptors 421, 422, 423, and 424. Delivery of one or more of the described HITMA agents to cancer cell 400 could occur in a number of ways. A first composition 430 may include a DNA vector that encodes the HITMA agents. First composition 430 may target surface receptor 421, and may be targeted for delivery to nucleus 405. The enclosed DNA vector, once in cancer cell 400, may exist transiently (e.g., not integrated into the genome) may be integrated at a site 431, either randomly or targeted, into genome 410. The expression of the HITMA agents coding sequence may be driven by a constitutively expressed promoter, an inducible promoter, or to provide further cancer specificity, a promoter that is active in the targeted cancer type/subtype.

In another example, second composition 435 may include mRNA that encodes one or more HITMA agents. Second composition 435 may bind to surface receptor 422, and may be targeted for delivery to endoplasmic reticulum 415 for translation into the HITMA agent peptide. In other examples, third composition 440 may be a virus or retrovirus that encodes the HITMA agents and is delivered to cell 400 via surface receptor 423. Fourth composition 445 includes the HITMA agent peptide itself, and may be targeted to nucleus 405 via surface receptor 424.

The mechanism of delivery of the HITMA agents, be it as peptides or nucleotide constructs, provides a further opportunity for increased selectivity and bioavailability for cancer cells. The HITMA agents may be encapsulated into liposomes, micelles, or specially designed nanoparticles that are preferentially taken up by cancer cells through a process called endocytosis, as shown at 450. Other methods that create physical gradients or alter biophysical properties such as convection-enhanced delivery, may be used to improve delivery of the composition, particularly to solid tumors. The availability of such delivery vehicles is typically greater for solid tumors through a mechanism called the “enhanced permeation and retention (EPR) effect”. These delivery vehicles may be further modified by the attachment of peptide ligands or antibodies that target cell surface receptors over expressed in cancer cells. Similarly, viruses and retroviruses can be targeted to these over expressed cell surface receptors.

Once in the cancer cell, the HITMA agents 450 may seek out and bind to the hypomethylated repetitive DNA sequences and/or histone moieties they were designed to target and create a DSB through the action of the strand break inducing domain. Because of the repetitive nature of the target sequence, a significant number of DSBs may occur. If the cancer cell's DNA repair machinery repairs a DSB, then the continued presence of the HITMA agents may continue inducing DSBs until the cell death pathway is triggered in the cancer cell. Since many cancers are already deficient in the DNA repair of DSBs, this makes them inherently more susceptible to the apoptosis-inducing effects of HITMA agents.

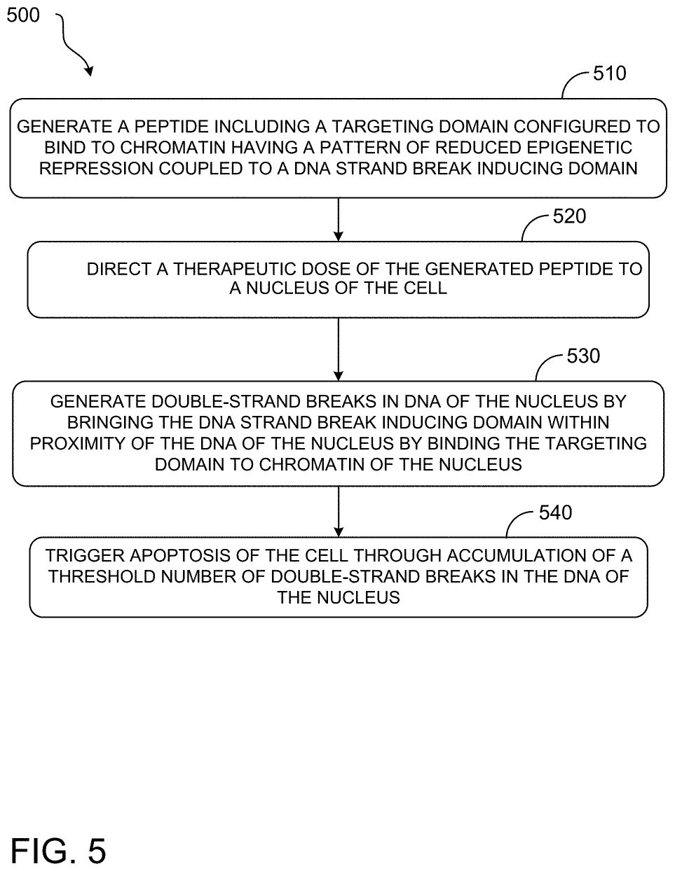

FIG. 5 shows an example method 500 for treating a mammalian cell having reduced epigenetic repression, in accordance with the current disclosure. As a non-limiting example, method 500 may be used to treat a human cell, or a plurality of human cells of a tumor-bearing human being.

At 510, method 500 includes generating a peptide including a targeting domain configured to bind to chromatin having a pattern of reduced epigenetic repression coupled to a DNA strand break inducing domain. In some examples, the peptide may be generated externally to the cell. Additionally or alternatively, method 500 may include providing a nucleotide construct encoding the peptide, and inducing production of the peptide within the cell, as described with regard to FIG. 4.

At 520, method 500 includes directing a therapeutic dose of the generated peptide to a nucleus of the cell. In examples wherein the peptide is generated externally to the cell, it may be packaged in a composition that includes a binding agent for one or more cell-surface receptors that target the nucleus of the cell. For examples wherein the peptide is generated within the cell, one or more targeting sequences may be included in the nucleotide construct that, when translated, direct the peptide to the nucleus.

At 530, method 500 includes generating double-strand breaks in DNA of the nucleus by bringing the DNA strand break inducing domain within proximity of the DNA of the nucleus by binding the targeting domain to chromatin of the nucleus. In some examples, method 500 may include generating a second peptide including a second DNA strand break inducing domain coupled to a second targeting domain configured to bind a second DNA sequence associated with the repetitive element, the second DNA sequence located within a threshold distance of the first DNA sequence on an opposite strand, and directing a therapeutic dose of the second generated peptide to the nucleus of the cell, as described with regard to FIG. 2 Continuing at 540, method 500 may include triggering apoptosis of the cell through accumulation of a threshold number of double-strand breaks in the DNA of the nucleus.

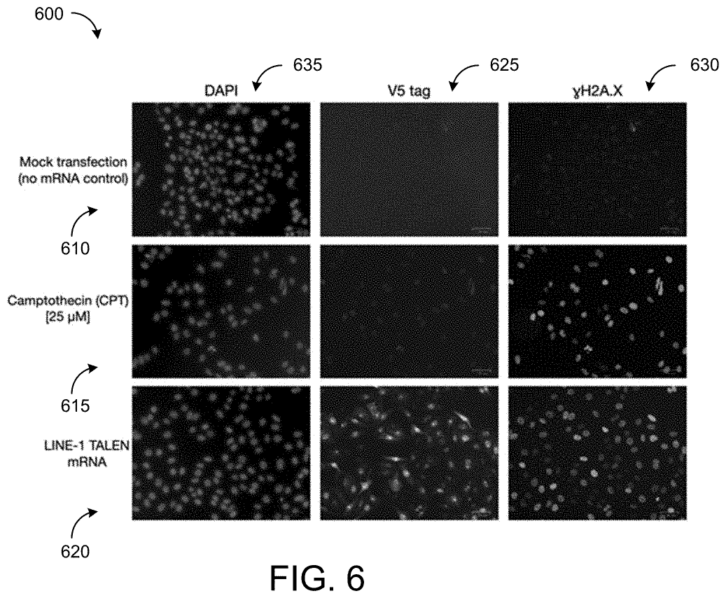

FIG. 6 shows experimental data 600 showing the induction of DNA damage through targeting of hypomethylated LINE-1 elements. In this example, the expression of TALEN(s) was designed to target the CpG-island of the long interspersed nuclear element-1 (LINE-1) repetitive element, thus provoking an induction of the histone variant H2A.X phosphorylated at the serine 139 reside (γH2A.X) in the SW480 colon cancer cell line. Loss of normal DNA methylation (aberrant hypomethylation) of LINE-1 elements is a feature of the SW480 colon cancer cell line (see, Kawakami et al., Cancer Sci. 2011 January; 102(1)166-74). The induction of γH2A.X is an indication of DNA damage (e.g., double-strand DNA breaks).

SW480 cells were either mock transfected (top row, 610), treated with camptothecin (middle row, 615) a known DNA double-strand break inducer, or transfected with LINE-1 TALEN(s) mRNAs with the V5 epitope tag encoded at their 5′ ends (bottom row, 620). After 24-hours, cells were fixed in 4% paraformaldehyde for 10 minutes at room temperature and then blocked/permeabilized by incubation for 60 minutes in blocking buffer (1× Phosphate Buffered Saline [PBS], 5% normal goat serum, 0.3% Triton X-100). After blocking, cells were incubated with antibodies against the V5 epitope (middle column, 625) and γH2A.X (Cell Signaling Technology) (right-hand column, 630) overnight at 4° C. and then washed with 1× PBS (3x-5 minutes). Cells were then incubated with Alexa Fluor® conjugated secondary antibodies (Cell Signaling Technology) for 60 min at room temperature in the dark, washed with 1× PBS (3x-5 minutes), and were then covered with Prolong Diamond Antifade reagent with DAPI (Thermo Fisher Scientific)—a nuclear DNA stain (left hand column, 635). Immunostained cells were observed with a fluorescence cell imager to visual and acquire images.

As seen at 640, cells transfected with LINE-1 TALEN(s) mRNAs exhibited similar, dramatic induction of yH2AX as did cells treated with camptothecin (645), thus suggesting that the LINE-1 TALEN was expressed, and that the expressed peptide did indeed induce apoptosis in SW480 cancer cells.

Additionally, phosphorylated H2A.X protein induction is seen both when “paired” LINE-1 TALENs are transfected into cells, but also when a single LINE-1 TALEN is used. LINE-1 elements may be both intensely repetitive and be clustered together in discrete parts of the nucleus. This clustering may bring threshold amounts of the FokI nuclease domains of single TALEN elements together to cause their activation.

In many examples, combination therapy approaches may be used that serve to enhance HITMA. The use of drugs (e.g. PARPi, DNA-PKi) or other approaches (e.g. siRNA, RNAi, CRISPR, etc.) to inhibit DSB DNA repair processes in the cell may enhance the apoptotic effect of HITMA. Indeed, evidence that DSBs are able to trigger apoptosis comes from studies on DNA repair defective cell lines. Cells defective in repairing DSBs by non-homologous end joining (NHEJ) or homologous recombination (HR) are sensitive to IR-induced cell killing, with NHEJ playing the dominant protective role. Other drugs may promote the apoptosis effect of HITMA by inhibiting anti-apoptotic proteins (e.g. [Bcl-2], inhibitor of apoptosis proteins, FLICE-inhibitory protein [c-FLIP]) and/or upregulation of proapoptotic proteins (e.g. BAX). Other drugs (e.g. 5-Azacytidine, 5-aza-2′-deoxycytidine, etc.) or approaches (e.g. siRNA, RNAi, CRISPR, etc.) may be used to inhibit the activity of the DNA methyltransferases (DNMTs) so as to reduce DNA methylation in cancer cells to enhance the HITMA effect. Similarly, molecules designed to target the repressive chromatin state may also be used to enhance accessibility and targeting of HITMA such as molecules that impact histone post-translational modification deposition (e.g., histone deacetylase inhibitors (HDACi), polycomb repressive complex inhibitors), recognition (e.g., bromodomain inhibitors), as well as molecules impacting chromatin structure (e.g., chromatin remodeling inhibitors).

While described predominantly with regard to human cancer treatment, HITMA may also be used in non-human mammals in veterinary medicine. Although aberrant DNA methylation has not been studied for cancers found in companion animals to the extent it has been in humans, similar aberrant methylation abnormalities occur in animal cancers. Repetitive sequences differ between species, therefore, species-specific HITMA-TALENs could be designed.

Specifically targeting and inducing DSBs in hypomethylated repetitive DNA sequences in cancer in order to induce apoptosis in cancer cells is both novel and non-obvious. It also has the advantages of being cancer-specific, with limited “off-target” effects expected in normal cells. Furthermore, a unique HITMA approach may be applied to each cancer type/subtype, creating a catalogue of HITMA therapeutics. The cancer-specificity of this approach can further be enhanced by the choice of delivery of the HITMA, by the promoter choice for the expression of the HITMA, and by the selection of complementary therapeutics for combination therapy.

Definitions (Adapted from Wikipedia)

“DNA methylation” describes the methylation of cytosine to form 5-methylcytosine occurs at the 5 position on the pyrimidine ring. In mammals, DNA methylation is almost exclusively found in CpG dinucleotides, with the cytosines on both strands being usually methylated.

“Repetitive DNA Sequences”—(also known as repeat sequences, repetitive elements, repeating units or repeats) are patterns of nucleic acids that occur in multiple copies throughout the genome. Major categories of repeated sequence or repeats include, but are not limited to: tandem repeats—copies which lie adjacent to each other, either directly or inverted; Satellite DNA—typically found in centromeres and heterochromatin; minisatellites—repeat units from about 10 to 60 base pairs, found in many places in the genome, including the centromeres; microsatellites—repeat units of less than 10 base pairs; this includes telomeres, which typically have 6 to 8 base pair repeat units; interspersed repeats (aka. interspersed nuclear elements); transposable elements; DNA transposons; retrotransposons; LTR-retrotransposons (HERVs); non LTR-retrotransposons; SINEs (Short Interspersed Nuclear Elements); LINEs (Long Interspersed Nuclear Elements); and SVAs.

“Transcription Activator-Like Effectors” (TALEs) include proteins secreted by Xanthomonas bacteria via their type III secretion system when they infect various plant species. These proteins can bind promoter sequences in the host plant and activate the expression of plant genes that aid bacterial infection. They recognize DNA sequences through a central repeat domain consisting of a variable number of ˜34 amino acid repeats.

“Transcription Activator-Like Effector Nucleases” (TALENs) include restriction enzymes that can be engineered to cut specific sequences of DNA. They may be made by fusing a TAL effector DNA-binding domain to a DNA cleavage domain (a nuclease which cuts DNA strands). TALEs can be engineered to bind to practically any desired DNA sequence, so when combined with a nuclease, DNA can be cut at specific locations.

“Apoptosis” is a form of programmed cell death that occurs in multicellular organisms. “Genotoxicity” describes the property of chemical agents that damages the genetic information within a cell causing mutations, which may lead to cancer. “Endonucleases” are enzymes that cleave the phosphodiester bond within a polynucleotide chain. “Homing Endonucleases” are a collection of endonucleases encoded either as freestanding genes within introns, as fusions with host proteins, or as self-splicing inteins (e.g., protein segments able to excise themselves and catalyze peptide binding of the remaining portions of the protein). They catalyze the hydrolysis of genomic DNA within the cells that synthesize them, but do so at very few, or even singular, locations. Repair of the hydrolyzed DNA by the host cell frequently results in the gene encoding the homing endonuclease having been copied into the cleavage site, hence the term ‘homing’ to describe the movement of these genes.

In one example, a composition of matter, comprises a nucleotide construct encoding a peptide, the peptide including at least: a targeting domain configured to bind to chromatin having a pattern of reduced epigenetic repression; and a DNA strand break inducing domain. In such an example, or any other example, the targeting domain is additionally or alternatively configured to bind to histone moieties not associated with DNA methylation. In any of the preceding examples, or any other example, the targeting domain is additionally or alternatively a methylation-sensitive DNA binding domain configured to bind to a first DNA sequence associated with a repetitive element, the DNA sequence having a cancer-specific hypomethylation pattern. In any of the preceding examples, or any other example, the first DNA sequence associated with a repetitive element is additionally or alternatively a long interspersed nuclear element (LINE) sequence. In any of the preceding examples, or any other example, the nucleotide construct additionally or alternatively encodes a second peptide, the second peptide comprising a second targeting domain configured to bind a second DNA sequence associated with the repetitive element, the second DNA sequence located within a threshold distance of the first DNA sequence on an opposite strand; and the DNA strand break inducing domain. In any of the preceding examples, or any other example, the DNA strand break inducing domain additionally or alternatively includes a nuclease domain. In any of the preceding examples, or any other example, the nuclease domain additionally or alternatively includes a FokI nuclease domain. In any of the preceding examples, or any other example, the DNA strand break inducing domain additionally or alternatively includes a methylation-sensitive nuclease domain. In any of the preceding examples, or any other example, the nucleotide construct is additionally or alternatively an mRNA construct. In any of the preceding examples, or any other example, the nucleotide construct is additionally or alternatively a DNA construct.

In another example, a method for treating a mammalian cell having reduced epigenetic repression, comprises generating a peptide including a targeting domain configured to bind to chromatin having a pattern of reduced epigenetic repression coupled to a DNA strand break inducing domain; directing a therapeutic dose of the generated peptide to a nucleus of the cell; generating double-strand breaks in DNA of the nucleus by bringing the DNA strand break inducing domain within proximity of the DNA of the nucleus by binding the targeting domain to chromatin of the nucleus; and triggering apoptosis of the cell through accumulation of a threshold number of double-strand breaks in the DNA of the nucleus. In such an example, or any other example, the method additionally or alternatively comprises providing a nucleotide construct encoding the peptide; and inducing production of the peptide within the cell. In any of the preceding examples, or any other example, directing a therapeutic dose of the generated peptide to a nucleus of the cell additionally or alternatively includes packaging the peptide in a composition that includes a binding agent for one or more cell-surface receptors that target the nucleus of the cell. In any of the preceding examples, or any other example, the targeting domain is additionally or alternatively configured to bind to histone moieties not associated with DNA methylation. In any of the preceding examples, or any other example, the targeting domain is additionally or alternatively a methylation-sensitive DNA binding domain configured to bind to a first DNA sequence associated with a repetitive element and having a cancer-specific hypomethylation pattern. In any of the preceding examples, or any other example, the method additionally or alternatively comprises generating a second peptide including a second DNA strand break inducing domain coupled to a second targeting domain configured to bind a second DNA sequence associated with the repetitive element, the second DNA sequence located within a threshold distance of the first DNA sequence on an opposite strand; and directing a therapeutic dose of the second generated peptide to the nucleus of the cell. In any of the preceding examples, or any other example, the nuclease domain additionally or alternatively includes a FokI nuclease domain.

In yet another example, a composition of matter, comprises a first peptide including a first nuclease domain coupled to a first methylation-sensitive DNA binding domain configured to bind to a first DNA sequence associated with a repetitive element and having a cancer-specific repetitive hypomethylation pattern; and a second peptide including a second nuclease domain coupled to a second methylation-sensitive DNA binding domain configured to bind to second DNA sequence at a threshold distance from the first DNA sequence on an opposite strand. In such an example, or any other example, the first and second nuclease domains additionally or alternatively include a FokI nuclease domain. In any of the preceding examples, or any other example, the first DNA sequence having a cancer-specific repetitive hypomethylation pattern is additionally or alternatively a long interspersed nuclear element (LINE) sequence.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A composition of matter, comprising:
a nucleotide construct encoding a peptide, the peptide including at least:
a targeting domain configured to bind to chromatin having a pattern of reduced epigenetic repression; and
a DNA strand break inducing domain.

2. The composition of matter of claim 1, wherein the targeting domain is configured to bind to histone moieties not associated with DNA methylation.

3. The composition of matter of claim 1, wherein the targeting domain is a methylation-sensitive DNA binding domain configured to bind to a first DNA sequence associated with a repetitive element, the DNA sequence having a cancer-specific hypomethylation pattern.

4. The composition of matter of claim 3, wherein the first DNA sequence associated with a repetitive element is a long interspersed nuclear element (LINE) sequence.

5. The composition of matter of claim 3, wherein the nucleotide construct further encodes a second peptide, the second peptide comprising
a second targeting domain configured to bind a second DNA sequence associated with the repetitive element, the second DNA sequence located within a threshold distance of the first DNA sequence on an opposite strand; and
the DNA strand break inducing domain.

6. The composition of matter of claim 1, wherein the DNA strand break inducing domain includes a nuclease domain.

7. The composition of matter of claim 6, wherein the nuclease domain includes a FokI nuclease domain.

8. The composition of matter of claim 1, wherein the DNA strand break inducing domain includes a methylation-sensitive nuclease domain.

9. The composition of matter of claim 1, wherein the nucleotide construct is an mRNA construct.

10. The composition of matter of claim 1, wherein the nucleotide construct is a DNA construct.

11. A method for treating a mammalian cell having reduced epigenetic repression, comprising:
generating a peptide including a targeting domain configured to bind to chromatin having a pattern of reduced epigenetic repression coupled to a DNA strand break inducing domain;
directing a therapeutic dose of the generated peptide to a nucleus of the cell;
generating double-strand breaks in DNA of the nucleus by bringing the DNA strand break inducing domain within proximity of the DNA of the nucleus by binding the targeting domain to chromatin of the nucleus; and
triggering apoptosis of the cell through accumulation of a threshold number of double-strand breaks in the DNA of the nucleus.

12. The method of claim 11, further comprising:
providing a nucleotide construct encoding the peptide; and
inducing production of the peptide within the cell.

13. The method of claim 11, wherein directing a therapeutic dose of the generated peptide to a nucleus of the cell includes packaging the peptide in a composition that includes a binding agent for one or more cell-surface receptors that target the nucleus of the cell.

14. The method of claim 11, wherein the targeting domain is configured to bind to histone moieties not associated with DNA methylation.

15. The method of claim 11, wherein the targeting domain is a methylation-sensitive DNA binding domain configured to bind to a first DNA sequence associated with a repetitive element and having a cancer-specific hypomethylation pattern.

16. The method of claim 15, further comprising:
generating a second peptide including a second DNA strand break inducing domain coupled to a second targeting domain configured to bind a second DNA sequence associated with the repetitive element, the second DNA sequence located within a threshold distance of the first DNA sequence on an opposite strand; and
directing a therapeutic dose of the second generated peptide to the nucleus of the cell.

17. The method of claim 11, wherein the nuclease domain includes a FokI nuclease domain.

18. A composition of matter, comprising:
a first peptide including a first nuclease domain coupled to a first methylation-sensitive DNA binding domain configured to bind to a first DNA sequence associated with a repetitive element and having a cancer-specific repetitive hypomethylation pattern; and
a second peptide including a second nuclease domain coupled to a second methylation-sensitive DNA binding domain configured to bind to second DNA sequence at a threshold distance from the first DNA sequence on an opposite strand.

19. The composition of matter of claim 18, wherein the first and second nuclease domains includes a FokI nuclease domain.

20. The composition of matter of claim 18, wherein the first DNA sequence having a cancer-specific repetitive hypomethylation pattern is a long interspersed nuclear element (LINE) sequence.
